SEQUENCE LISTING (10 sequences)

SEQ ID NO: 1 - DNA, 1762 bp, Rattus norvegicus
agaaacgctccgagtttcggggtcctccctgcgcctggctcccagaacttcccggagtgcggcccaggcccgggtagagggggaggggaccaagcgtcctggtcgcgggaaggcgggatgcagagctgcgaactgctacgcgctactgagccgggaacgaggagccaccggccacacgaccttctgcaggcgccttgcaccatgcacccccggcgccgcggctcctgcagccttgccgcgtcccccagggacagcactgaacgcttccagccgccgatcggagctggacctgcgtcgccgcgcctctcagggagccccggccgcggtgctggagcaaacacccgcctctaccagccgcggggccgccggcatggatggatgaggacgcccatgccagaagtgcgcggaactcggacaaacttttccagcggccccgcggccgccacccgagcctcgtttccgctccgcaccgcgtccggcggccgctgctgcccgcgatgctagccgcactgctgggcggcggcggagcccgctcggggagcttgccgggcgccttgctgtgcctgatggcgctgctgcagttgctgggctcggcgccgcggggctcggggctggcgcacggccgccgcctcatctgctggcaggcgctgctgcagtgtcagggagagccggactgcagctacgcctacagccagtacgccgaggcgtgtgcgccggtgctcgcacagcgcggcggagctgatgctcctgggcccgtcggcgccttcccctcctcggccgcgtccttctcgccgcgctggcgatgcccgagccactgcatctcggcgctcatccagctcaaccacacgcgccgcgggcccgcgctggaggactgcgactgcgcgcaggacgaacactgcaggtccaccaagcgcgccatcgagccctgtctgccgcgcaccagcagcgtgggcccgggcgcgggagcgggctcggtcatgggctgcaccgaggcccggcggcgctgcgaccgggacagccgctgcaacctggcactcggccgctatctagcctactgcggcaagcttttcaacggtctccgccgcaccgacgagtgccgcgcggtcatcgaggacatgctggcggtgcccaaggcggcgctgctcaacgactgtgtgtgcgatgggctggagaggcccatctgcgagtcggtcaaggagaacatggcccgcctgtgcttcggcccagatgcgagcaacggtccgggcagcagcggctcggacgggggcctagacgactactacgacgaagaatatgatgacgagcagcgcgctggggccgcgggcggcgagcagcccctggacgacgacgacggccttgcgcgcccgggcggcggcgctgctggggcaggcggacgcggggacttgccccacgggccggggcgcaggagcagcagcagcggcagtggaggccactgggcgacccgaagctcctggaccccgtttgcctgttttctgctgctcctgctgcttgggtcccacttgtagcccccgcgcccctgctgcaggctactccaggccggtacgcgcgacccagctgattcccgagcagggcacctgtggctcggagaccggagatgatcaggagcactgacccacactctccgcgggtagacagggcccagcccgtaccgcaactaccatgctcggcccttttctgtgcggttttccaggctgcagataaaaccggccgatctatactgccggctctaatctgcagaatt

SEQ ID NO: 2 - protein, 383 aa, Rattus norvegicus
MDEDAHARSARNSDKLFQRPRGRHPSLVSAPHRVRRPLLPAMLAALLGGGGARSGSLPGALLCLMALLQLLGSAPRGSGLAHGRRLICWQALLQCQGEPDCSYAYSQYAEACAPVLAQRGGADAPGPVGAFPSSAASFSPRWRCPSHCISALIQLNHTRRGPALEDSDCAQDEHCRSTKRAIEPCLPRTSSVGPGAGAGSVMGCTEARRRCDRDSRCNLALGRYLAYCGKLFNGLRRTDECRAVIEDMLAVPKAALLNDCVCDGLERPICESVKENMARLCFGPDASNGPGSSGSDGGLDDYYDEEYDDEQRAGAAGGEQPLDDDDGLARPGGGAAGAGGRGDLPHGPGRRSSSSGSGGHWATRSSWTPFACFLLLLLLGSHL

SEQ ID NO: 3 - DNA, 1762 bp, Rattus norvegicus
aattctgcagattagagccggcagtatagatcggccggttttatctgcagcctggaaaaccgcacagaaaagggccgagcatggtagttgcggtacgggctgggccctgtctacccgcggagagtgtgggtcagtgctcctgatcatctccggtctccgagccacaggtgccctgctcgggaatcagctgggtcgcgcgtaccggcctggagtagcctgcagcaggggcgcgggggctacaagtgggacccaagcagcaggagcagcagaaaacaggcaaacggggtccaggagcttcgggtcgcccagtggcctccactgccgctgctgctgctcctgcgccccggcccgtggggcaagtccccgcgtccgcctgccccagcagcgccgccgcccgggcgcgcaaggccgtcgtcgtcgtccaggggctgctcgccgcccgcggccccagcgcgctgctcgtcatcatattcttcgtcgtagtagtcgtctaggcccccgtccgagccgctgctgcccggaccgttgctcgcatctgggccgaagcacaggcgggccatgttctccttgaccgactcgcagatgggcctctccagcccatcgcacacacagtcgttgagcagcgccgccttgggcaccgccagcatgtcctcgatgaccgcgcggcactcgtcggtgcggcggagaccgttgaaaagcttgccgcagtaggctagatagcggccgagtgccaggttgcagcggctgtcccggtcgcagcgccgccgggcctcggtgcagcccatgaccgagcccgctcccgcgcccgggcccacgctgctggtgcgcggcagacagggctcgatggcgcgcttggtggacctgcagtgttcgtcctgcgcgcagtcgcagtcctccagcgcgggcccgcggcgcgtgtggttgagctggatgagcgccgagatgcagtggctcgggcatcgccagcgcggcgagaaggacgcggccgaggaggggaaggcgccgacgggcccaggagcatcagctccgccgcgctgtgcgagcaccggcgcacacgcctcggcgtactggctgtaggcgtagctgcagtccggctctccctgacactgcagcagcgcctgccagcagatgaggcggcggccgtgcgccagccccgagccccgcggcgccgagcccagcaactgcagcagcgccatcaggcacagcaaggcgcccggcaagctccccgagcgggctccgccgccgcccagcagtgcggctagcatcgcgggcagcagcggccgccggacgcggtgcggagcggaaacgaggctcgggtggcggccgcggggccgctggaaaagtttgtccgagttccgcgcacttctggcatgggcgtcctcatccatccatgccggcggccccgcggctggtagaggcgggtgtttgctccagcaccgcggccggggctccctgagaggcgcggcgacgcaggtccagctccgatcggcggctggaagcgttcagtgctgtccctgggggacgcggcaaggctgcaggagccgcggcgccgggggtgcatggtgcaaggcgcctgcagaaggtcgtgtggccggtggctcctcgttcccggctcagtagcgcgtagcagttcgcagctctgcatcccgccttcccgcgaccaggacgcttggtcccctccccctctacccgggcctgggccgcactccgggaagttctgggagccaggcgcagggaggaccccgaaactcggagcgtttct

SEQ ID NO: 4 - protein, 131 aa, Rattus norvegicus
HCRSTKRAIEPCLPRTSSVGPGAGAGSVMGCTEARRRCDRDSRCNLALGRYLAYCGKLFNGLRRTDECRAVIEDMLAVPKAALLNDCVCDGLERPICESVKENMARLCFGPDASNGPGSSGSDGGLDDYYD

SEQ ID NO: 5 - DNA, 15 bp, Artificial Sequence
  Description of Artificial Sequence Synthetic oligonucleotide
tcctcatccatccat

SEQ ID NO: 6 - DNA, 26 bp, Artificial Sequence
  Description of Artificial Sequence Synthetic primer
gaattcgagaaacgctccgagtttcg

SEQ ID NO: 7 - DNA, 37 bp, Artificial Sequence
  Description of Artificial Sequence Synthetic primer
ggatccagttttaatacagtttatacgacgtaccagg

SEQ ID NO: 8 - DNA, 15 bp, Artificial Sequence
  Description of Artificial Sequence Synthetic oligonucleotide
tcctcatcgatggta

SEQ ID NO: 9 - DNA, 15 bp, Artificial Sequence
  Description of Artificial Sequence Synthetic oligonucleotide
gagctggtgttccat

SEQ ID NO: 10 - DNA, 15 bp, Artificial Sequence
  Description of Artificial Sequence Synthetic oligonucleotide
gtagttgtccatggt

(19) United States
(12) Patent Application Publication
(10) Pub. No.: US 2008/0085861 A1
(43) Pub. Date: Apr. 10, 2008

(54) GAS1 POLYPEPTIDES

(76) Inventors: Walter Herman Maria Louis LUYTEN, Beerse (BE); Jose Ramon Naranjo, Madrid (ES); Britt Mellstrom, Madrid (ES)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/691,714

(22) Filed: Mar. 27, 2007

Related U.S. Application Data
(63) Continuation of application No. 10/069,495, filed on Feb. 20, 2002, now abandoned.

(30) Foreign Application Priority Data
Aug. 24, 1999 (EP) 99306702.4

(51) Int. Cl. C07K 14/00; A61K 31/7088; C07K 16/00; C12N 15/09; C12N 15/63; C12Q 1/02; C12N 15/11; C12N 1/00; A61K 38/16

(52) U.S. Cl. 514/12; 435/243; 435/29; 435/320.1; 435/440; 514/44; 530/387.9; 536/23.1; 536/24.1

(57) ABSTRACT

There is disclosed a method of inhibiting the lethal effect of expressing an otherwise lethal protein in a cell, said method comprising: (a) providing a cell, tissue or organism having (i) a nucleotide sequence encoding a Gas1 protein, or a functional equivalent, derivative or bioprecursor thereof, which is capable of inducing apoptosis in said cell and (ii) a further nucleotide sequence encoding a protein which is otherwise lethal to said cell in itself or in response to a lethal stimulus in the presence of Gas1; (b) inhibiting function and/or expression of said Gas1 protein or functional equivalent, derivative or bioprecursor thereof; and (c) expressing said sequence encoding said otherwise lethal protein.

The present invention is concerned with methods of identifying compounds capable of preventing or accelerating Gas1 mediated cell death.

The Gas1 gene encodes a membrane protein which has been associated with the G0 phase of proliferative arrest and cell cycle exit in rat fibroblasts deprived of serum. Based upon its antiproliferative effects and its functional dependency to p53, Gas1 has also been associated with antitumour like activity. (Schneider et al., 1988 Genes specifically expressed at growth arrest in mammalian cells, Cell 54:787-793; Del Sal et al, 1992, the growth arrest specific gene, Gas1, is involved in growth suppression, Cell 70:593-607; Del Sal et al, 1994. Structure, function and chromosome mapping of the growth suppressing human homologue of the murine Gas1 gene, Proc. Natl. Acad. Sci. USA 91; 1848-1852). The structural conformation of the Gas1 protein deduced from the amino acid sequence thereof, indicates the presence of two transmembrane segments. Except for a hypothetical RGD domain (Arginine-Glycine-Aspartic acid) which is known to interact with integrines, Gas1 does not show any described domain that relates to its cell cycle arrest function. Recently, mRNA Gas1 induction during involution of the prostate, mammary gland and the ovarian luteal body, as a result of castration, lactation and birth delivery arrest, respectively, has been observed (Jaggi et al., 1996, Regulation of a Physiological Apoptosis Mouse mammary Involution. J. Davy Sci. 79: 1074-1084).

From expression studies, the present inventors have surprisingly found that Gas1 overexpression induces cell death in various cell types, such as, neurons and neuroblastoma cell lines and that Gas1 is responsible for the induction of apoptotic activity in a cell.

Based upon the surprising relationship of Gas1 expression and apoptosis the present inventors have developed methods to study the effects of expressing proteins in a cell which are normally lethal to the cell by inhibiting expression or activity of either the Gas1 protein or a protein in the signal transduction pathway of which Gas1 is a component. These methods can be further applied to identifying compounds which inhibit or enhance the expression of those otherwise lethal proteins. An assay has also been developed to identify compounds which are capable of preventing or accelerating Gas1 mediated cell death.

Therefore in accordance with a first aspect of the invention there is provided, a method of inhibiting the lethal effect of expressing an otherwise lethal protein in a cell, said method comprising (a) providing a cell, tissue or organism having (i) a nucleotide sequence encoding a Gas1 protein, or a functional equivalent, derivative or bioprecursor thereof, which is capable of inducing apoptosis in said cell and (ii) a further nucleotide sequence encoding a protein which is otherwise lethal to said cell in itself or in response to a lethal stimulus in the presence of Gas1; (b) inhibiting function and/or expression of said Gas1 protein or functional equivalent, derivative or bioprecursor thereof or a protein in the apoptotic pathway of which Gas1 is a component; and (c) expressing said sequence encoding said otherwise lethal protein.

Thus, advantageously, it is now possible, by inhibiting the function or expression of the biological mediator of cell death to study proteins which are normally lethal when expressed in a cell. These methods can also be used to identify compounds which can function as enhancers/inhibitors of expression or activity of the otherwise lethal proteins and which has not been possible hitherto.

Therefore, according to a second aspect of the present invention there is provided a method of identifying compounds which inhibit or enhance expression or activity of proteins which are otherwise lethal to a cell, tissue or organism said method comprising (a) providing a cell, tissue or organism comprising a nucleotide sequence encoding a Gas1 protein or a functional equivalent, derivative or bioprecursor thereof, which is capable of inducing apoptosis in said cell, and ii) a further sequence encoding a protein which is otherwise lethal to said cell in itself or in response to a lethal stimulus in the presence of Gas1; (b) inhibiting function and/or expression of said Gas1 protein or a functional equivalent, derivative or bioprecursor thereof or a protein in the apoptotic pathway of which Gas1 is a component; (c) expressing said sequence encoding said otherwise lethal protein; (d) contacting said cell with a compound to be tested; and (e) monitoring the effect of said compound on said otherwise lethal protein compared to an identical cell which has not been contacted with said compound.

Preferably, the inhibition of activity or expression of the Gas1 protein occurs by providing a nucleic acid molecule, such as an antisense molecule, which is capable of hybridising to mRNA in the cell corresponding to or complementary to Gas1 DNA under stringent conditions, to prevent expression thereof. The nucleic acid molecule in addition to possessing antisense activity, may in some embodiments possess ribozyme or DNAzyme activity.

The methods of the present invention, therefore, involve inhibiting the function or expression of a Gas1 protein in vivo using, for example, antisense technology. Antisense technology can be used to control gene expression through triple-helix formation or antisense DNA or RNA, both of which methods are based on binding of a polynucleotide to DNA or RNA. For example, the part of the DNA sequence coding for the mature protein of the present invention is used to design an antisense RNA oligonucleotide of from 10 to 50 base pairs in length. A DNA oligonucleotide is designed to be complementary to a region of the gene involved in transcription (triple-helix—see Lee et al. Nucl. Acids. Res., 6:3073 (1979); Cooney et al., Science, 241:456 (1988); and Dervan et al., Science, 251:1360 (1991), thereby preventing transcription and the production of Gas1. The antisense RNA oligonucleotide hybridises to the mRNA in vivo and blocks translation of an mRNA molecule into the mature protein. Thus, an animal having expression of the Gas1 protein inhibited may be utilised as a model for expression of otherwise lethal proteins in accordance with the methods of the invention and for identifying potential therapeutic agents capable of inhibiting or enhancing expression or activity of the otherwise lethal proteins.

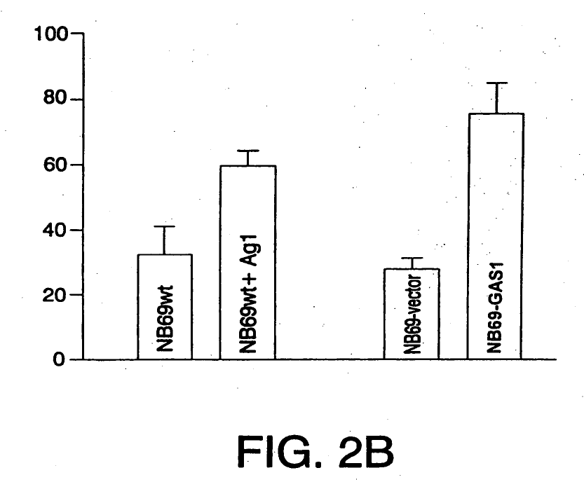

Preferably, the antisense molecule comprises a specific Gas1 antisense oligonucleotide (SEQ ID 5) which the present inventors have confirmed as being able to block the transduction process of the Gas1 protein and, as described in more detail below, involves a total blocking of the NMDA induced neuron death phenomenon in primary neuron cultures (survival practically of 100%, 25 mM Ag1 column in FIG. 2a) or by staurosporin (200 nM) (FIG. 2b). Alternatively, a sequence complementary to the nucleotide sequence encoding Gas1 as identified in SEQ ID No. 3 may be used to prevent expression of the Gas1 protein.

In an even further embodiment it is also possible to inhibit Gas1 expression or activity by, for example, inhibiting expression or activity of a protein which is involved in the pathway of which Gas1 is a component. Thus, using the methods described it is possible to prevent the apoptotic properties of Gas1 proteins being realised either by inhibiting, for example, the signal transduction pathway upstream or downstream of Gas1 DNA to prevent the apoptotic response. It has also been identified that induction of Gas1 leads to a simultaneous increase in calcium concentration within the cell. In the method according to the invention a suitable stimulus can be applied which is lethal to a cell, to induce transcription of the Gas1 protein.

In one embodiment of the methods described herein, the otherwise lethal protein may be provided on a suitable expression vector. Suitable vectors are well known to those of skill in the art. Similarly, the sequence of the further nucleotide sequence encoding said otherwise lethal protein may be provided in an expression vector under the control of suitable regulatory control elements. Such lethal proteins include, for example, glutamate receptors such as any of the type 1 to 8 metabotropic receptors, or NMDA, AMPA or kainate receptors. Alternatively, the lethal protein may comprise a highly expressed recombinant protein which can frequently be toxic to a cell within which it is expressed. Thus, the present invention also, advantageously, provides a system to enable harvesting of highly expressed recombinant proteins.

The nucleic acid molecule utilised in accordance with the methods of the invention to inhibit expression of the Gas1 protein may be provided as an oligonucleotide, which is transformed or transfected into the cell, using techniques well known to those of skill in the art. Alternatively, the sequence may be encoded by a suitable sequence provided in a vector, which is transformed or transfected into the cell, tissue or organism.

Any compounds identified are also encompassed within the scope of the present invention. Thus, a further aspect of the invention provides a compound identifiable as an inhibitor or an enhancer of expression or activity of an otherwise lethal protein according to the invention. Such compounds may, for example, be included, in pharmaceutical compositions together with a pharmaceutically acceptable carrier, diluent or excipient therefor.

Compounds identified as enhancers of expression or activity may be used in the manufacture of a medicament for treating a disease condition mediated at least in part by underexpression or reduced activity of said otherwise lethal protein or a protein in the pathway of which said otherwise lethal protein is a component. Likewise compounds identified as inhibitors of expression or activity may be used in the manufacture of a medicament for treating a disease condition mediated at least in part by overexpression or reduced activity of said otherwise lethal protein or a protein in the pathway of which said otherwise lethal protein is a component. Preferably, the disease condition to be treated comprises any of a neurological disorder, a cardiovascular disorder, an autoimmune disorder, a neuroendocrine disorder or cancer.

These compounds can be used to prevent cell death in neurological disorders, such as for example, Parkinson's disease, Alzheimer's disease, Huntington's disease, amyotrophic lateral sclerosis, cerebellar ataxias, spinal muscular atrophies, etc.; stroke; head trauma; epilepsies; cardiovascular disorders, for example, post infarction, etc.; neuroendocrine disorders, for example, pituitary necrosis, etc.; autoimmune diseases, for example, multiple sclerosis, etc.; and any pathological process which is at least partially mediated by Gas1.

Furthermore, the present invention relates to a method of producing an antagonist or agonist of Gas1 according to the invention comprising the steps of any one of the above described screening methods; and additionally (i) synthesizing the compound obtained or identified in said method or a physiologically acceptable analog or derivative thereof in an amount sufficient to provide said antagonist or agonist in a therapeutically effective amount to a patient; and/or (ii) combining the compound obtained or identified in said method or an analog or derivative thereof with a pharmaceutically acceptable carrier.

The compounds isolated by the above methods also serve as lead compounds for the development of analog compounds. The analogs should have a stabilized electronic configuration and molecular conformation that allows key functional groups to be presented to the Gas1 protein in substantially the same way as the lead compound. In particular, the analog compounds have spatial electronic properties which are comparable to the binding region, but can be smaller molecules than the lead compound, frequently having a molecular weight below about 2 kD and preferably below about 1 kD. Identification of analog compounds can be performed through use of techniques such as self-consistent field (SCF) analysis, configuration interaction (CI) analysis, and normal mode dynamics analysis. Computer programs for implementing these techniques are available; e.g., Rein, Computer-Assisted Modeling of Receptor-Ligand Interactions (Alan Liss, New York, 1989). Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art; see also supra. Furthermore, peptidomimetics and/or computer aided design of appropriate derivatives and analogues can be used.

A further aspect of the invention comprises a method of monitoring the severity of a disease condition mediated by cellular apoptosis in a cell, tissue or organism comprising measuring the level of transcription, expression or activity of a Gas1 protein or a functional equivalent, derivative or bioprecursor thereof in said cell or tissue or organism.

The present inventors have also now, advantageously, identified the sequence of the rat Gas1 polypeptide and which has never before been fully characterised. Accordingly, a further aspect of the present invention comprises a nucleic acid molecule encoding a rat Gas1 protein or a functional equivalent, derivative or bioprecursor thereof, comprising an amino acid sequence according to sequence ID No. 2. Preferably, the nucleic acid molecule is a DNA molecule and even more preferably a cDNA molecule, which in a preferred embodiment comprises the sequence of nucleotides according Sequence ID No. 1. Alternatively, the invention provides in a further aspect, a nucleic acid molecule encoding a protein capable of inducing apoptosis in a cell, comprising an amino acid according to Sequence ID No. 4 or a nucleic acid molecule which is complementary thereto.

The invention also provides an antisense molecule capable of hybridising to a nucleic acid molecule according to the invention under conditions of high stringency.

Stringency of hybridisation as defined herein refers to conditions under which polynucleic acids are stable. The stability of hybrids is reflected in the melting temperature (Tm) of the hybrids. Tm can be approximated by the formula:
81.5° C.−16.6(log 10[Na⁺]+0.41(% G&C)−600/l
wherein 1 is the length of the hybrids in nucleotides. Tm decreases approximately by 1-1.5° C. with every 1% decrease in sequence homology.

Preferably, the antisense molecule comprises the sequence of nucleotides according to Sequence ID No. 5.

Advantageously, the nucleic acid molecule according to the invention may be used to express the Gas1 protein according to the invention, in a host cell or the like using an appropriate expression vector.

An expression vector according to the invention includes vectors capable of expressing DNA operatively linked to regulatory sequences, such as promoter regions, that are capable of effecting expression of such DNA fragments.

Regulatory elements required for expression include promoter sequences to bind RNA polymerase and transcription initiation sequences for ribosome binding. For example, a bacterial expression vector may include a promoter such as the lac promoter and for transcription initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or assembled from the sequences described by methods well known in the art. Thus, an expression vector refers to a recombinant DNA or RNA construct, such as a plasmid, a phage, recombinant virus or other vector that upon introduction into an appropriate host cell result in expression of the DNA or RNA fragments. Appropriate expression vectors are well known to those skilled in the art and include those that are replicable in eukaryotic cells and/or prokaryotic cells and those that remain episomal or those which integrate into the host cell genome.

The antisense molecule capable of hybridising to the nucleic acid according to the invention may be used as a probe or as a medicament or alternatively in a pharmaceutical composition, by preventing expression of a Gas1 protein. Advantageously, the antisense molecule according to the invention may be used as a drug, or in the preparation of a drug for the treatment of the previously described pathological processes. The expression vector including said antisense molecule according to the present invention may be used advantageously in vivo, such as in gene therapy (Matteucci & Wagner, Nature 384, Supp 7:20-22 (1996); Whitesell et al. Proc. Natl. Acad. Sci. USA 90:4665-4669 (1993); Wahlestedt, C. Trends Pharmacol. Sci. 15:42-46 (1994)).

Nucleic acid molecules according to the invention may be inserted into the vectors described in an antisense orientation in order to provide for the production of antisense RNA. Antisense RNA or other antisense nucleic acids may be produced by synthetic means.

A further aspect of the invention comprises the host cell transformed, transfected or infected with the expression vector according to the invention, which cell preferably comprises a eukaryotic cell and more preferably a mammalian cell.

Incorporation of cloned DNA into a suitable expression vector for subsequent transformation of a cell and subsequent selection of the transformed cells is well known to those skilled in the art as provided in Sambrook et al (1989) Molecular Cloning, A Laboratory manual, Cold Spring Harbour Laboratory Press.

A further aspect of the present invention comprises a nucleic acid molecule having at least 15 nucleotides of the nucleic acid molecule according to the invention and preferably from 15 to 50 nucleotides. These sequences may, advantageously be used as probes or primers to initiate replication or the like. Such nucleic acid molecules may be produced according to techniques well known in the art, such as by recombinant or synthetic means. They may also be used in diagnostic kits or devices or the like for detecting for the presence of a nucleic acid according to the invention. These tests generally comprise contacting the probe with a sample under hybridising conditions and detecting for the presence of any duplex formation between the probe and any nucleic acid in the sample.

According to the present invention these probes may be anchored to a solid support. Preferably, they are present on an array so that multiple probes can simultaneously hybridize to a single biological sample. The probes can be spotted onto the array or synthesised in situ on the array. (See Lockhart et al., Nature Biotechnology, vol. 14, December 1996 A Expression monitoring by hybridisation into high density oligonucleotide arrays≅. A single array can contain more than 100, 500 or even 1,000 different probes in discrete locations.

Nucleic acid molecules according to the invention may also be produced using such recombinant or synthetic means, such as, for example, using PCR cloning mechanisms which generally involve making a pair of primers, which may be from approximately 10 to 50 nucleotides to a region of the gene which is desired to be cloned, bringing the primers into contact with mRNA, cDNA, or genomic DNA from a human cell, performing a polymerase chain reaction under conditions which bring about amplification of the desired region, isolating the amplified region or fragment and recovering the amplified DNA. Generally, such techniques as defined herein are well known in the art, such as described in Sambrook et al (Molecular Cloning: a Laboratory Manual, 1989).

The nucleic acids or oligonucleotides according to the invention may carry a revealing label. Suitable labels include radioisotopes such as ³²p or ³⁵S, enzyme labels or other protein labels such as biotin or fluorescent markers. Such labels may be added to the nucleic acids or oligonucleotides of the invention and may be detected using known techniques per se.

Further provided by the present invention is a transgenic cell, tissue or organism comprising a transgene capable of expressing the rat Gas1 protein according to the invention. The term “transgene capable of expression” as used herein means any suitable nucleic acid sequence which leads to expression of a Gas1 protein having the same function and/or activity of a rat Gas1 protein according to the invention. The transgene may include, for example, genomic nucleic acid or synthetic nucleic acid including cDNA, integrated into the chromosome or in an extrachromosomal state.

Preferably, the transgene comprises a vector according to the invention, which vector includes a nucleic acid molecule encoding said rat Gas1 protein, or a functional fragment of said nucleic acid molecule. A “functional fragment≅ of said nucleic acid should be taken to mean a fragment of the gene or cDNA encoding said rat Gas1 or a functional equivalent thereof, which fragment is capable of being expressed to produce a functional rat Gas1 protein according to the invention.

In accordance with the present invention, a defined nucleic acid includes not only the identical nucleic acid but also any minor base variations including in particular, substitutions in bases which result in a synonymous codon (a different codon specifying the same amino acid residue) due to the degenerate code in conservative amino acid substitutions. The term “nucleic acid molecule” also includes the complementary sequence to any single stranded sequence given regarding base variations.

The invention provides for the rat Gas1 proteins themselves, encoded by the nucleic acid molecules according to the invention. Preferably, the Gas1 protein comprises the sequence of amino acids according to the sequence of amino acids of Sequence ID No. 2. In a further aspect of the invention comprises a protein capable of inducing apoptosis in a cell comprising an amino acid sequence according to Sequence ID No. 4 or a functional equivalent, derivative or bioprecursor thereof.

A “functional equivalent” as defined herein should be taken to mean a rat Gas1 protein that exhibits all of the growth properties and functionality associated with rat Gas1 protein. A “derivative” as defined herein is intended to include a polypeptide in which certain amino acids have been altered or deleted or replaced with other amino acids and which polypeptide retains the biological activity of Gas1 according to the invention and/or which polypeptide can react with antibodies raised using Gas1 according to the invention as the challenging antigen.

Encompassed within the scope of the present invention are hybrid and modified forms of rat Gas1, including fusion proteins and fragments. The hybrid and modified forms include, for example, when certain amino acids have been subjected to some modification or replacement, such as for example, by point mutation yet which modifications still result in a protein which retains the biological activity of Gas1, according to the invention. Specific nucleic acid sequences can be altered by those of skill in the art to produce a protein exhibiting the same or substantially properties to Gas1 of the invention.

A defined protein, polypeptide or amino acid sequence according to the invention includes not only the identical amino acid sequence but isomers thereof in addition to minor amino acid variations from the natural amino acid sequence including conservative amino acid replacements (a replacement by an amino acid that is related in its side chains). Also included are amino acid sequences which vary from the natural amino acid but result in a polypeptide which is immunologically identical or similar to the polypeptide encoded by the naturally occurring sequence.

Proteins or polypeptides according to the invention further include variants of such sequences, including naturally variants which are substantially homologous to said proteins or polypeptides. In this context, substantial homology is regarded as a sequence which has at least 70%, and preferably 80%, 90% or 95% amino acid homology with the proteins or polypeptides encoded by the nucleic acid molecules according to the invention.

Substantial homology should be taken to mean that the nucleotide and amino acid sequences of the Gas1 of the invention display a certain degree of sequence identity. Preferably they share an identity of at least 30%, preferably 40%, more preferably 50%, still more preferably 60%, most preferably 70%, and particularly an identity of at least 80%, preferably more than 90% and still more preferably more than 95% is desired with respect to the nucleotide or amino acid sequences depicted in Seq. ID Nos. 1 to 4, respectively. A preferred method for determining the best overall match between a query sequence (a sequence of the present invention) and a subject sequence, also referred to as a global sequence alignment, can be determined using, for example, the FASTDB computer program based on the algorithm of Brutlag et al. (Comp. App. Biosci. 6 (1990), 237-245.) In a sequence alignment the query and subject sequences are both DNA sequences. An RNA sequence can be compared by converting U's to T's. The result of said global sequence alignment is in percent identity. Further programs that can be used in order to determine homology/identity are described below and in the examples. The sequences that are homologous to the sequences described above are, for example, variations of said sequences which represent modifications having the same biological function, in particular encoding proteins with the same or substantially the same receptor specificity, i.e. binding specificity. They may be naturally occurring variations, such as sequences from other mammals, or mutations. These mutations may occur naturally or may be obtained by mutagenesis techniques. The allelic variations may be naturally occurring allelic variants as well as synthetically produced or genetically engineered variants.

Antibodies to the Gas1 protein according to the invention may advantageously be prepared by techniques which are well known to those of skill in the art.

The therapeutic or pharmaceutical compositions of the present invention can be administered by any suitable route known in the art including for example intravenous, subcutaneous, intramuscular, transdermal, intrathecal or intracerebral or administration to cells in ex vivo treatment protocols. Administration can be either rapid as by injection or over a period of time as by slow infusion or administration of slow release formulation. For treating tissues in the central nervous system, administration can be by injection or infusion into the cerebrospinal fluid (CSF).

Gas1 protein, the antisense molecules or indeed the compounds identified as enhancers or inhibitors of activity or expression of Gas1 or the otherwise lethal proteins may be used in the form of a pharmaceutical composition, which may be prepared according to procedures well known in the art. Preferred compositions include a pharmaceutically acceptable vehicle or diluent or excipient, such as for example, a physiological saline solution. Other pharmaceutically acceptable carriers including other non-toxic salts, sterile water or the like may also be used. A suitable buffer may also be present allowing the compositions to be lyophilized and stored in sterile conditions prior to reconstitution by the addition of sterile water for subsequent administration. Incorporation of the aforementioned compounds or antisense molecules, for example, into a solid or semi-solid biologically compatible matrix may be carried out which can be implanted into tissues requiring treatment.

The carrier can also contain other pharmaceutically acceptable excipients for modifying other conditions such as pH, osmolarity, viscosity, sterility, lipophilicity, solubility or the like.

Pharmaceutically acceptable excipients which permit sustained or delayed release following administration may also be included.

The compounds identified in accordance with the method of the invention may be administered orally. In this embodiment they may be encapsulated and combined with suitable carriers in solid dosage forms which would be well known to those skilled in the art.

As would be well known to those of skill in the art, the specific dosage regime may be calculated according to the body surface area of the patient or the volume of body space to be occupied, dependent upon the particular route of administration to be used. The amount of the composition actually administered will, however, be determined by a medical practitioner, based on the circumstances pertaining to the disorder to be treated, such as the severity of the symptoms, the composition to be administered, the age, weight, and response of the individual patient and the chosen route of administration.

The invention may be more clearly understood from the following examples and accompanying figures wherein:

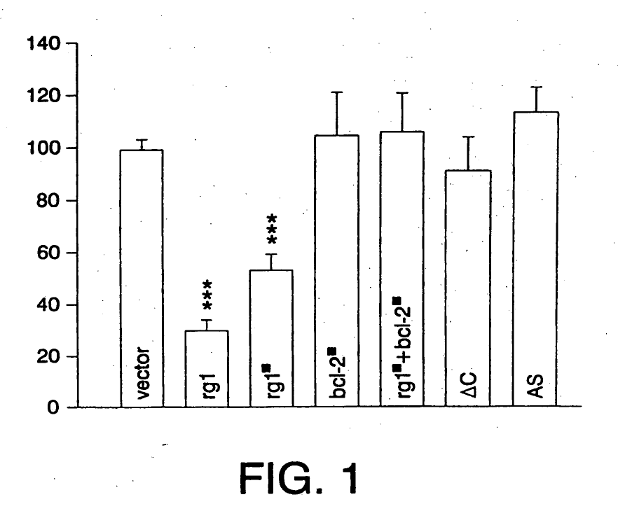

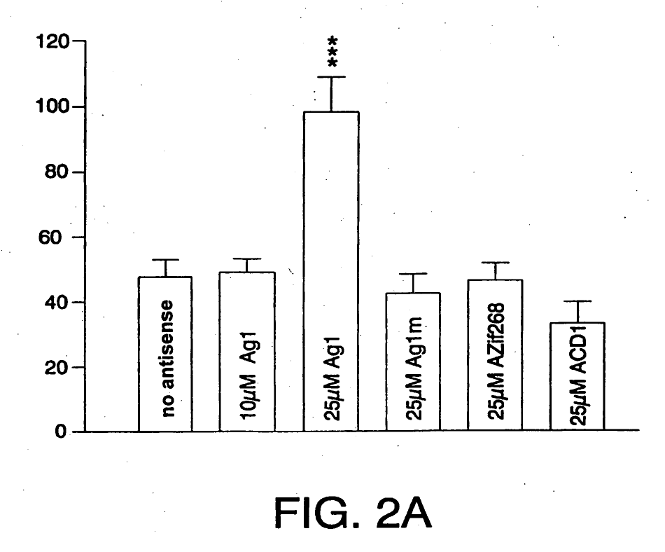

FIG. 1 is a graphic representation of induction of cell death in hippocampal neurons by transient overexpression of Gas1. Quantitative analysis of the effect of rat Gas1 overexpression is represented as % survival 24 hours after transfection of the different expression vectors and the combination of expression vectors, that is showed under each column. Values are referred to 100% survival which corresponds to transfection with empty vector pcDNA3 as indicated in the first column. Overexpression of Gas1 resulted in a reduction of neuronal survival to 30% (1.5 μg of Gas1, rg1) or 50% (0.75 μg of Gas1, rg1*). Overexpression of the C-terminal truncated form of Gas1 (ΔC) did not have a significant effect on cell survival at 24 hr. Cotransfection of Gas1 and Bcl-2 resulted in the total protection of the neurons (rg1*+Bcl-2*). While overexpression of Bcl-2 alone (1.5 μg) or the complementary form of Gas1 (AS, 1.5 μg) did not result in survival values different from the control. Results are expressed as mean +/−SEM. Statistical analysis was performed by Student's t-test. *** denotes p>0.001, and other treatment groups were statistically non-significant (p<0.5).

FIG. 2 is a graphic representation of the effects of protection against cell death by an antisense Gas1 oligonucleotide and by complementary Gas1 RNA.

A.—Antisense Gas1 (25 μM Ag1) blocks NMDA-induced neuronal death in cortico-hippocampal cultures. The viability of mature cortico-hippocampal cultures was analyzed 24 hr after 500 mM NMDA exposure. Pretreatment with the different antisense oligonucleotides at the indicated concentrations was done 24 hr before the exposure to NMDA. Results are expressed as mean +/−SEM. Statistical analysis was performed by Student's t-test. *** denotes p>0.001, and other treatment groups were statistically non-significant (p<0.5).

B.—Antisense Gas1 (25 μM Ag1) prevents the reduction in survival induced by 200 nM staurosporine in NB69 wild type cells (NB69 wt). Columns 1 and 2. Moreover, stable expression of complementary Gas1 RNA in NB69-Gas1 cells protects from staurosporine-induced neuronal death as compared to NB69 mock cells stably transfected with the empty vector. Columns 3 and 4. The viability was analyzed 24 hr after exposure to staurosporine (200 nM) in all the cases. Results are expressed as mean +/−SEM. Statistical analysis was performed by Student's t-test. *** denotes p>0.001, and other treatment groups were statistically non-significant (p<0.5).

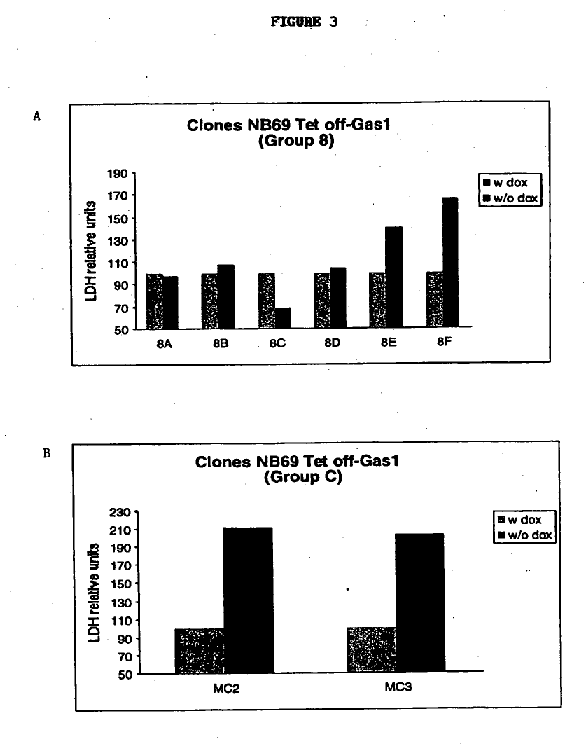

FIG. 3 is an illustration showing increase in LDH levels in the culture media at different times after doxicycline removal from indicated clones. FIG. 3A shows the result for subclones from group 8 and FIG. 3B shows the result for subclones from group C.

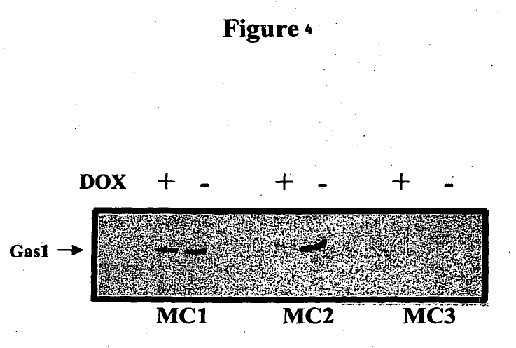

FIG. 4 is an illustration of the results obtained form a Western blot analysis showing the induction of Gas-1 immunoreactivity after doxicycline removal from indicated clones.

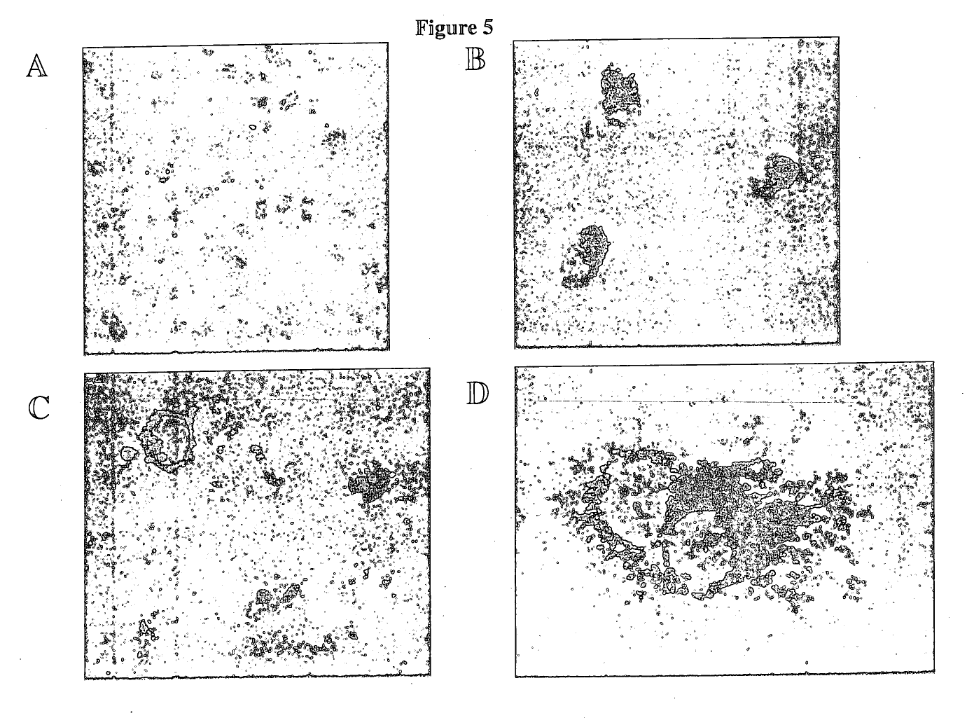

FIG. 5 is an illustration of the results obtained from staining with Annexin-V Fluos (green) before (A) and 24 h. after (B) removal of doxicycline in cultures of NB69-MC3 cells. Propidium iodine staining could be observed at later times (48 h. or more) after doxicycline removal (C) and (D).

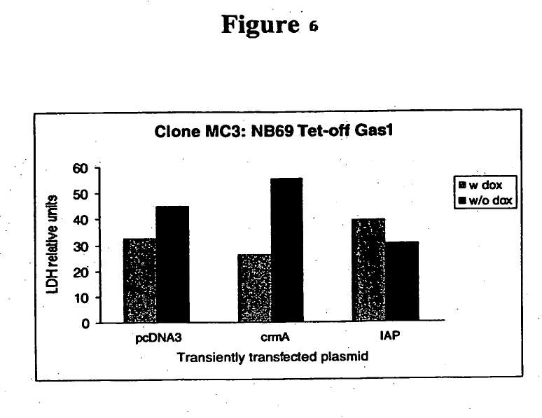

FIG. 6 is a diagrammatic representation of results showing reduced LDH release to the culture media after doxicycline removal in C3 cells transiently transfected with IAP.

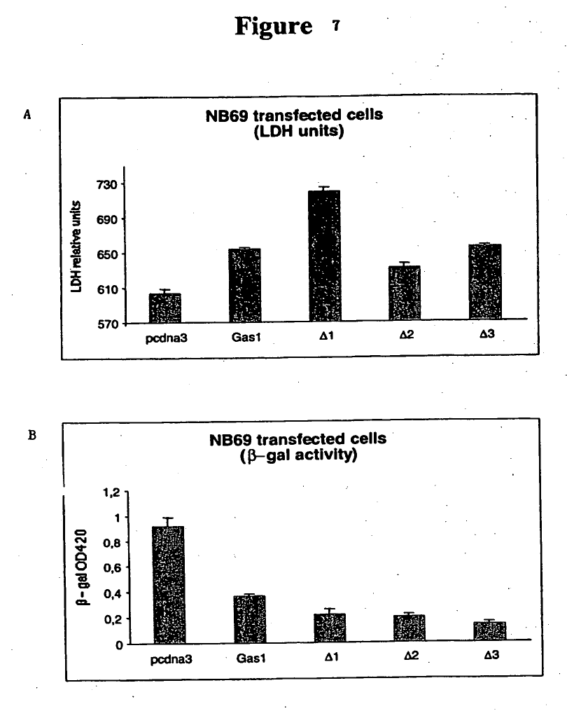

FIG. 7 is a diagrammatic representation of results showing increase in LDH levels in the culture media and reduced beta-galactosidase activity after transient transfection of wtGas1 or mutants Δ1 to Δ3.

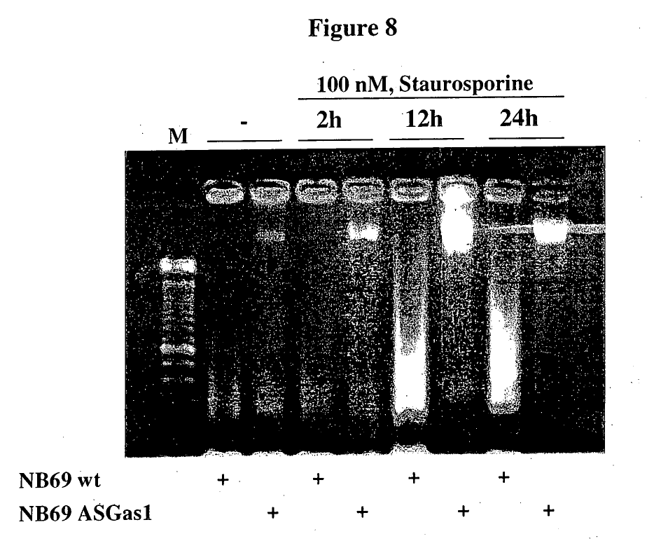

FIG. 8 is an illustration of results showing fragmentation of chromosomal DNA in NB69 cells treated with 200 nM staurosporine is prevented by stable overexpression of antisenseGas1. Standard ladder DNA is shown in lane 1 (M).

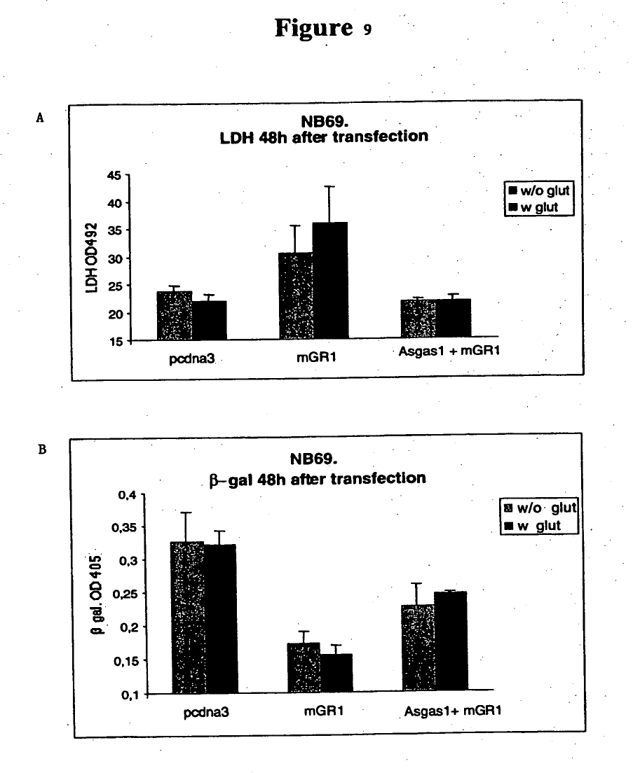

FIG. 9 is a diagrammatic representation of the increase in LDH release and the reduction in beta-galactoside activity after overexpression of human mGluR1 in NB69 cells is blocked by cotransfection of antisense Gas1.

### EXAMPLE 1

### Cloning Rat Gas1

50,000 primary phages from a cDNA library prepared from cortico-hippocampal primary cultures 6 hr after a brief (5 min.) exposure to 500 mM of NMDA were differentially screened. Primary cortico-hipocampal cultures were prepared from E17 rat fetuses essentially as described (Choi, 1987). Neuronal cultures were used for the experiments after twelve days in culture. To prepare the libraries, poly-A+mRNA was prepared from cortico-hippocampal control or NMDA treated cultures using the microFastTrack kit from Invitrogen. Oligo-dT primed cDNA was cloned in 1ZAP (Stratagene) following manufacturer's instructions. Fifty thousand phages were differentially screened, using ³²P-labeled cRNA probes derived from poly-A+ RNA isolated from control cultures (− probe), or from cultures 6 hr after the NMDA treatment (+ probe) and the use of the AMV reverse transcriptase (25 U, BRL). For differential screening of the library prepared from treated cultures, each pair of nitrocellulose filters upon which phages have been transferred are subjected to probe (+) or probe (−) hybridization for 20 hours at 421 C in a hybridization buffer that contains 50% formamide, 10% dextrane sulfate, 4×SSC, 0.1% SDS, Tris-HCl 10 mM, pH 7.4, 1×denhardt's, 50 (g/ml) and salmon sperm and the corresponding probe 106 dpm/ml. Then the filters are washed for 30 min. at room temperature in 2×SSC, 0.1% SDS, twice for 30 min. At 42° C. in 2×SSC, 0.1% SDS and twice for 30 min. At 55° C. in 0.2×SSC, 0.1% SDS. After autoradiographic exposure for five days, the hybridization signals obtained for each pair of filters hybridized with probe (+) or probe (−) are compared, looking for the differential presence of hybridization signal in filters (+) in relation to (−). In this way a 714 bp clone initially named pCHN-414 is obtained, and once sequenced, it showed homology with the untranslated 3′ region of the Gas1 human and rat gene. This indicates that the pCHN-414 clone corresponds to the rat Gas1 gene, which is confirmed in the following experiments.

A cDNA fragment containing the complete coding sequence of rat Gas1 was obtained following PCR using rat genomic DNA as template. The 5′ primer was derived from the mouse sequence (Del Sal et al., 1992) at position 49-66 (5′-GAATTCGAGAAACGCTCCGAGTTTCG-3′, SEQ ID NO:6). The sequence of the 3′ primer was obtained from the rat clone (5′-GGATCCAGTTTTAATACAGTTTATACGACGTACCAGG-3′, SEQ ID NO:7), at a position corresponding to 2449-2483 in the mouse sequence. Flanking EcoRI and BamHI sites in the 5′ ends of the oligonucleotides were designated for cloning purposes. The DNA was then amplified by the polymerase chain reaction using Tli DNA polymerase (Promega) for 40 cycles. The timing for each cycle was as follows: 1 min. at 94° C., 1 min. at 60° C. and 2 min. at 72° C. The cycling was preceded by 2 min. denaturing period at 94° C. and followed by a 7 min. extension at 72° C. A 2.4 kb PCR product was obtained, restricted by EcoRI, cloned into pCDNA3 (Invitrogene) and completely sequenced at both strands using Thermo stable Sequenase and the conditions suggested by the manufacturer (Amersham-Pharmacia).

### EXAMPLE 2

### Gas1 Gene Transfection Induces Neuronal Death

To assess the effect of the Gas1 protein on neuronal viability, pGas1 was cotransfected together with a β-galactosidase expression vector that served as a marker of transfected neurons using liposomes. For optimal liposome-mediated transfection efficiency these experiments were performed in serum-free conditions using primary cultures of hippocampal neurons. Culture of hippocampal neurons was performed in chemically defined medium as described (Ohsawa, et al, 1993. Response of embryonic hippocampal neurons in culture to neurotrophin-3, brain-derived neurotrophic factor and basic fibroblast growth factor. Neurosci. 57, 67-77.). Hippocampal neurons (3 DIV, 2×10⁶ cells per 35 mm dish) received 2 μg total amount of plasmid DNA containing 0.5 μg of β-galactosidase expression vector (pCH110, Pharmacia) and 1.5 μg of pCDNA3 vector (InVitrogene), empty or containing the Gas1 coding sequence in the 5′ to 3′ orientation (rg1), the Gas1 coding sequence in the complementary orientation (3′ to 5′, AS) or the truncated Gas1 (ΔC, The C-terminal truncated expression vector for Gas1 contains amino acids 1 to 229 and was prepared from the rg1 plasmid by subcloning of a HindIII/EcoRI fragment into pcDNA3). For the protection by Bcl-2, 0.5 μg of rg1 and 0.75 μg of pBcl-2 were used together with 0.5 μg of pCH110. A ratio of 1:2 DNA: Transfectam (Promega) was used for the preparation of liposomes. Three hours after transfection, the liposome-containing medium was replaced with fresh medium. To assess the number of transfected neurons, the cultures were fixed with 2.5% glutaraldehyde 24 hr after the transfection and X-Gal staining was performed as suggested (Promega). The experiments were repeated 3 to 5 times, performed in quadruplicates and each plate was counted by three independent investigators. X-Gal positive neurons present in 20 fields of each plate were counted and averaged. The average of X-Gal positive neurons in control cultures was taken as 100% survival for each experiment. The transfection efficiency was similar for the different plasmids, for different preparations of a given plasmid and among experiments, as assayed following a similar protocol but 16 hr after transfection, a time point at which cell death has not started. The transfection efficiency accounted for approximately 1% of the total cell population, and showed little variation between the different experimental groups and between experiments. At 24 hr after transfection, the total number of X-Gal positive neurons in cultures transfected with rg1 was dramatically reduced. The survival after Gas1 overexpression was decreased in a concentration-dependent manner to 30% and 50% when transfected with 1.5 and 0.75 μg of pGas1, respectively (FIG. 1). In contrast, the overexpression of the complementary strand of Gas1 (AS), did not affect the number of viable X-Gal positive cells at 24 hr after transfection (FIG. 1). These results associate the overexpression of Gas1 with neuronal death and indicate that high levels of Gas1 protein are sufficient to trigger a death process that is accompanied by profound alterations in morphology.

### EXAMPLE 3

### Blocking of Translation of the Gas1 Protein by Gas1 Antisense Oligonucleotide or by Gas1 Complementary Chain Overexpression Protects Against Excitotoxic Death or Death Induced by Staurosporine.

NMDA-induced excitotoxicity was analyzed in cortico-hippocampal cultures maintained for 12-14 DIV similar to those already described in Example 1. To apply the excitotoxic insult, the cultures were rinsed twice with Locke's solution without Mg2+ and exposed to 500 mM NMDA (Sigma) for 5 min. in the same Locke's solution or to Locke's solution alone as control. NMDA was washed out, the Mg2+ concentration was restored by 2 changes of Locke's +Mg2+ solution, and the original medium was replaced. Phase-bright bipolar cells, taken to represent living neurons, were counted 24 hr after the NMDA exposure. Alternatively, the vital staining method (Jones and Senft, 1985 An improved method to determine cell viability by simultaneous staining with fluorescein diacetate-propidium iodide. J Histochem Cytochem 33:77-79) was used to assess the extent of neuronal death and the protection by antisense oligonucleotide treatments. Similar results were obtained using either method. The vital staining method was used to prepare FIG. 2a.

A 15-mer Gas1 antisense oligonucleotide 5′-TCCTCATCCATCCAT-3′ SEQ ID NO:5, (AG1) spanning the start site of translation (underlined) was used to specifically block Gas1 translation. As a negative control a 15-mer oligonucleotide with nucleotide substitutions 5′-TCCTCATCGATGGTA-3′, SEQ ID NO:8 (AG1mut) was used and the Gas1-unrelated antisense oligonucleotides for cyclin D1:5′-GAGCTGGTGTTCCAT-3′, SEQ ID NO:9(Matsushime et al., 1991 Colony-stimulating factor 1 regulates novel cyclins during the G1 phase of the cell cycle Cell 65:701-713) and for zif268: 5′-GTAGTTGTCCATGGT-3′ SEQ ID NO:10 (Milbrandt, 1987 A nerve growth factor-induced gene encodes a possible transcriptional regulatory factor. Science 238:797-799). All oligonucleotides were protected at each end by two phosphorothioate groups. The oligonucleotides were added to the culture medium 24 hr before the induction of neuronal death by NMDA, at the concentrations indicated in the figures. Experiments were done in triplicate and repeated as least five times.

Staurosporine-induced neuronal death was assayed in human neuroblastoma NB69 cells cultured in DMEM/HAM F12 medium supplemented with 10% fetal calf serum, 2 mM glutamine and 50 μg/ml gentamycin. Wild-type or stably transfected NB69 cells (2×104 cells/35 mm dish) were exposed to 100 nM staurosporine (RBI). Phase-bright cells without signs of membrane or neurite degeneration were counted 24 hr later. Experiments were done in triplicate and repeated as least five times. Stably transfected NB69 cells that overexpress Gas1 in the antisense orientation (NB69-Gas1) were prepared by the calcium phosphate precipitation method followed by two weeks of geneticin selection. Control cells were prepared in parallel by transfection with pCDNA3 vector alone (NB69-vector). Expression of the constructs in the cell clones were analyzed by northern blot analysis, and the clones having the highest expression were chosen for the experiments. Stable tranfectants were kept in the presence of 0.3 mg/ml of geneticin, which was removed before plating for the experiments.

Neuron death by overstimulation of excitatory amino acid receptors is a well characterized phenomenon that receives the generic denomination of excitotoxicity. Excitotoxicity is a complex process that involves significant changes in the cell cycle gene expression. The detection of various of these genes was carried out in the course of differential screening of primary culture rat cortico-hippocampal neuron library at an excitotoxic concentration (500 mM) of N-methyl D-aspartic-NMDA acid (Choi, 1987, Ionic dependence of glutamate neurotoxicity in cortical cell culture. J. Neurosci 7:369-379). Cloning and sequence determination of one of them identified it as part of the rat homologue of the Gas1 gene described above in humans. Subsequently, the complete rat Gas1 gene was cloned and sequenced, as referred to below whose nucleotide sequence is attached as SEQ ID 1, obtaining from it the amino acid sequence of the rat Gas1 protein whose sequence is attached as SEQ ID 2.

Subsequently, the increase of 8 to 10 times the Gas1 mRNA expression after 6 hours of administering a pulse of NMDA in primary cortico-hippocampal cell cultures in contrast to control cells was confirmed by Northern blot. On the other hand, Gas1 mRNA induction has not been observed in pure primary culture of hippocampal astroglia after exposure to NMDA or glutamate (data not shown), suggesting that Gas1 overexpression occurs in neurons as part of the gene response during NMDA induced neuronal degeneration.

Hence, Gas1 expression in experimental models where neuron death was induced by means of different harmful stimuli has been evaluated. First of all, intraperitoneal administration of kainic acid was used, an experimental model that has demonstrated that it produces acute and delayed neuron death in different cerebral areas that include the hippocampus, olfactory cortex, thalamus and amygdala ((Schwob et al, Widespread patterns of neuronal damage following systemic or intracerebral injections of kainic acid: a histological study. Neurosci 5:991-1014; 1980; Sperk et al., Kainic acid-induced seizures: neurochemical and histopatological changes. Neurosci 10:1301-1315; 1983). Northern blot analysis demonstrated that Gas1 expression in control rat brain (without administering kainic acid) was very low in accordance with previous data (Del Sat et al., 1994). However, strong induction of Gas1 expression in hippocampus and olfactory cortex after intraperitoneal administration of 10 mg/kg of kainic acid was observed. Already detectable induced Gas1 mRNA levels after kainic acid appear 6 hours after the beginning of treatment and were maintained for at least 10 days. In order to reveal Gas1 induction and to confirm neurone localization thereof in situ hybridization after administering kainic acid was performed. A RNA specific for Gas1 monocatenary probe revealed, 24 hours after administration of kainic acid, signs of intense hybridization in isolated neurons, being evident in the pyramidal layer of the CA1 area of the hippocampus. Similar results were observed after provoking an experimental ischemic process in gerbil brain by transient occlusion of both carotids, giving rise to rapid degeneration of a numerous population of pyramidal neurons of the CA1 layer of the hippocampus. It was observed that this massive neuron death was preceded by a significant rise of Gas1 mRNA and Gas1 protein levels (evaluated by immunocytochemistry) in those neurons. Likewise, it was also observed that neuron loss in all the cerebral cortex that is produced in rats in the perinatal period after exposure to ionizing radiation is preceded by high induction of Gas1 mRNA. All these results suggest that Gas1 is involved in regulation of neuron death by excitotoxicity.

In a subsequent step to show a direct relationship between Gas1 gene expression and neuron death, hippocampal neurons were transfected with the pcDNA3 eukaryotic expression vector containing the Gas1 (rg1) gene. The experiments were carried out in primary cultures of rat fetus hippocampal neurons that were transfected with different amounts of the Gas1 expression vector, evaluating a posteriori the survival of these cells (FIG. 1). Hence, it was observed that transfection of cells with 1.5 μg of rg1 construct caused a significant reduction of survival, obtaining levels of survival lower than 40%, with regard to the control group (transfected with the empty expression vector), or with regard to the group of cells transfected with the Gas1 (SEQ ID 3) complementary or antisense sequence (Antisense(AS) column, see FIG. 1). The effect depends on the amount of Gas1 construct transfected and thus when transfection took place with half the amount (rg1*) the reduction of survival was more limited although still significant (FIG. 1).

On the other hand, this reduction of survival of neurons transfected with Gas1 (rg1*column) is completely blocked by coexpression in these same neurons with the Bcl-2 protein (rg1*+Bcl-2 column), Bcl-2 being the antiapoptotic protein prototype (FIG. 1). On the other hand, these experiments with rat Gas1 were repeated in NB69 human cells and in NIH3T3 rat fibroblasts, obtaining similar results. Besides, NB69 human neuroblastoma and 3T3 rat fibroblast cells were transfected with human Gas1, observing cell death of the NB69 and 3T3 cells.

Initially, Gas1 expression has been related to the G0 phase of cell cycle arrest in rat fibroblasts deprived of serum (Del Sal et al., 1992). However, the present invention shows that Gas1 overexpression in cortico-hippocampal neurons induces neuron death, acting as a protein with apoptosis inductive activity. Up to now it had been described that Gas1 protein was a cell membrane protein and as of its amino acid sequence the presence of two transmembrane segments had been proposed and a hypothetical RGD domain that may interact with integrin, the only domain described that relates its structure to its arrest function of the cell cycle (Schneider et al.). The present inventors have succeeded in overexpressing the Gas1 protein by means of a prokaryotic vector (pTrcHis) in bacteria for subsequent purification in sufficient amounts to carry out protein studies. Hence, it was observed in experiments of artificial bilayers that the purified Gas1 protein is capable of inserting in and forming cationic channels and that said capacity may be linked to neuron death induction. Aside from the already described location of this protein in the cell membrane we have located the presence of Gas1 in the perinuclear and mitochondrial membrane. It must be pointed out that Bcl-2 and Bax, proteins related to the cell cycle, are also located in the mitochondrial membrane and are capable of forming ionic channels. This common location of these proteins makes compatible a possible functional interaction of Gas1 with Bax type proapoptotic proteins and Bcl-2 and Bcl XL antiapoptotic proteins. The present inventors have also been able to demonstrate, by mutational analysis of the RGD domain, that this is not implied in neuron apoptotic activity of the Gas1 protein. On the contrary, we have delimited the neuron death inductive capacity in an amphipathic domain in the Gas1 terminal carboxyl region, located between the amino acids 174 and 304 of the protein (SEQ ID 4), that does not coincide with any of the previously hypothesized domains.

In order to confirm the relevance of this region of the protein in the lethal capacity of Gas1 hippocampal neurons were transfected with a mutated Gas1 construction in that region (^{a}C column, FIG. 1). As seen in FIG. 1, survival of these neurons is similar to that verified in the control group (vector column) and is significantly higher than that of neurons transfected with wild type Gas1 (rg1 and rg1* columns).

Thanks to the exact knowledge of the structure activity relationship in Gas1 polypeptide there is now a rational base to understand the lethal action of Gas1 as well as for the development of molecules capable of blocking the Gas1 polypeptide function or Gas1 gene expression with which to create new therapeutic tools to reduce or eliminate cell death. For this purpose, overexpression of Gas1 protein in human neuroblastoma cells or in rat fetus neurons by means of transfection with liposomes of the Gas1 eukaryotic expression vector was used. This may be constitive expression (pcDNA3) in transient expression but it may also be an inducible expression (pIND) that permits preparation of stable lines that will express Gas1 after induction of the expression vector for example with muristerone in the case of pIND. To evaluate the phenomenon of death one can resort to morphological criteria by means of transfection with b-galactosidase or by means of GFP (green fluorescent protein) or to biochemical criteria of degradation, detection of nucleic acids or release of dehydrogenase lactate into the medium, or any other indication of cellular lysis. The specific application of this invention is its use as a screening system of molecule collections in the search for products active in blocking Gas1 polypeptide function or Gas1 gene expression and therefore, providing a protective action against cell death. These screening systems form part of the present invention.

Transgenic models provide a useful model for assaying and testing drugs for their effectiveness and safety in the treatment of the above described diseases. Also, by means of the use of specific tissue promoters or cellular phenotype promoters Gas1 expression can be achieved in target tissues and cells for a better and more specific testing. An advantage of the invention is that potential Gas1 inhibitors and/or blocking agents can be readily tested in an in vivo model that closely mimics a human by the use of a rat gene for Gas1.

Furthermore, a specific Gas1 antisense oligonucleotide (SEQ ID 5) was verified as being able to block the translation of Gas1 protein and this involves a total blocking of the NMDA induced neuron death phenomenon in primary neuron cultures (survival practically of 100%, 25 mM Ag1 column in FIG. 2a) or by staurosporin induced death (200 nM) (FIG. 2b).

Upon observing that the stable expression of high levels of Gas1 complementary messenger in the NB69-Gas1 cell lines makes these cells more resistant to external harmful stimuli, it was possible to use these cells for forced expression of lethal genes that would induce cell death in cells but that would not cause death in these Gas1 expressing protected lines. An example of the possible application of this protected system (NB69-Gas1) is the overexpression of the glutamate type I metabotropic receptor (mGluR-I). This provides a system which permits the study of the pharmacology of mGluR-I and to screen possible agonist or antagonist molecules of this receptor. The example presented for mGluR-I is only one of the multiple possibilities of using Gas1 inhibition to achieve stable expression systems for genes that are normally detrimental to the survival of a cell. Other lethal proteins to evaluate by means of this expression system comprise, for example, ionotropic glutamate receptors including NMDA-receptors, AMPA receptors, kainate receptors with their various subunits and variants: metabotropic glutamate receptors (including subtype 1 to 8); other excitatory amino acid receptors (e.g. taurine); cytokine receptors; chemokine receptors; mono amine receptors; peptide receptors; enzymes such as kinases, caspases; and any other protein in signal transduction cascade mediating cell death.

The present inventors have also developed and produced polyclonal rabbit antibodies and rat hybridomas which can produce Gas1 monoclonal antibodies. These antibodies have been used in the immunocytochemical and Western blot studies that are described in this invention for the purpose of evaluating the expression of Gas1 protein. It is to be emphasized that these antibodies may be used for analytical purposes to purify said polypeptide, for in vitro diagnosis or for therapeutic purposes of disorders, especially those that imply cell death and neurodegeneration, where this polypeptide is expressed.

Effect of Functional Inhibition of Gas1 on Neuronal Viability.

Preparation of a human Gas1 inducible expression vector using the Tet-Off system. Firstly, an attempt was made to obtain double transfectants RXR-pINDhGas1 for ecdysone-induced expression of hGas-1 in the human neuroblastoma cell line NB69. This approach proved to be difficult and no clones showing a reasonable growth rate could be selected. In most cases, double transfectants presented long neuritic processes corresponding to a phenotype of terminally differentiated neurons, had insignificant incorporation of ³H-thymidine and a notorious accumulation of apoptotic bodies could be observed in the cultures. Attempts to reduce the percentage of serum in the media or to culture under serum-free conditions did not significantly improve the situation. It may be that a very high expression of the RXR receptor in NB69 primary clones NB69-RXR4 and NB69-RXR7 leads to a ligand-independent activation of the pINDhGas1 vector which could be the origin of the problem. Accordingly, NB69-RXR clones expressing low or moderate levels of the RXR receptor were tried. Thus, NB69 cells were cotransfected with RXR and pINDhGas1 and a double selection with G418 and Zeocin was performed. Three weeks after transfection clones could be observed. However, the growth rate of these clones was again very slow and abundant apoptotic bodies could be distinguished surrounding and on the top of each clone. In view of these negative results the ecdysone-based approach was abandoned in favour of a different inducible system performed in this case the Tet-Off system from (available from Clontech). Two complete rounds of selection were performed. NB69 cells were initially stably transfected with the TetOff plasmid using G418 for selection. Twelve clones were chosen in each round, 1 to 12 in the first and A to L in the second, for checking of expression levels of the TetOff repressor by transient transfection with reporter TRE-Luc again available from Clontech. After this analysis, clones 3, 8 and C were finally selected for cotransfection with the inducible expression vector TRE-hGas1 (a plasmid including the hGas1 sequence cloned in the pTRE2 vector available from Clontech) and the selection vector TK-hyg (Clontech). Selection was carried out for two weeks using 2 mg/ml of hygromycine in the presence of 1 μg/ml of doxicycline to keep the repressor TetOff bound to the TRE promoter in the TRE-hGas1 construct. Twelve subclones were again chosen from each of the three groups (3, 8 and C) and Gas1-induced cell death was assayed after doxicycline removal using the release of lactate dehydrogenase (LDH) as an index of cell damage (Cytotox kit, Promega). Positive clones were further analyzed for the inducibility of Gas1 after doxicycline removal by western blot using the monoclonal antibody G8F9-1 specific for Gas1. Some of these results are summarized in FIGS. 3 and 4. As a result, five TetOff-inducible clones were finally selected: 8d, 8e, 8f, C2 and C3.

Analysis of the molecular mechanism of Gas1-induced cell death. To assess the cell death mechanism after induction of Gas1 by doxicycline removal we first processed each of the clones using staining for annexin-V (Annexin-V Fluos, kit, Roche) as a hallmark of apoptosis and the staining with propidium iodine as an indication of a necrotic mechanism. As shown in FIG. 5 using clone C3, an intense green fluorescence in the outer membrane was observed soon after doxicycline removal, while at later times some of the cells developed an intense nuclear red fluorescence due to the entrance of the intercalating agent propidium iodine (FIGS. 5C and D). These results indicate that the mechanism of death involves a purely apoptotic process, at least at the early stages. Because of this the effect of caspase inhibition on Gas1-induced cell death after doxicycline removal was tested. For that, clones C2 and C3 were transfected with different expression vectors for crmA, IAP and p35, three caspase inhibitors with distinct specificity for different caspases. In these experiments it was found that overexpression of IAP, a caspase inhibitor of non-viral origin, selectively blocked Gas1-induced cell death after doxicycline removal (FIG. 6). This result indicates that inhibition of the pro-caspase 9 or the effector caspases 3, 6 and 7 are involved in the death process triggered by Gas1. Further studies using more selective caspase inhibitors as well as specific substrates for the different caspases are needed to identify the caspase directly responsible of the death after Gas-1 induction.

Molecular Analysis of Death-Related Domains in Gas1.

Domain analysis of the amphipathic α-helix region of Gas1. As a result of the frame-shift strategy it was possible to delineate a domain encompassing amino acids 174 to 279 in rat Gas1 involved both in the channel activity as well as in the death-inducing properties of Gas1. To more precisely map the specific residues responsible for these two biological activities deletion of discrete fragments of Gas1 within the 174-279 region was performed. Three deletion mutants have, so far, been prepared and analyzed Gas1 Δ1 to Δ3. Transient cotransfection experiments of each construct with an expression vector for lacZ was performed in NB69 cells and release of LDH and beta-galactosidase activity was measured as an index of cell death and viability, respectively. In all cases, a lethal effect similar to wtGas1 was observed. The results of a representative experiment are shown in FIG. 7.

Effect of Knockout of Gas1 on Neuronal Viability.

Stable expression of antisense Gas1 RNA in NB69 cells: effects on survival. To develop a cellular model highly resistant to the overexpression of potentially toxic proteins i.e. metabotropic glutamate receptors, we selected NB69 clones stably transfected with an expression vector for Gas1 cloned in the antisense orientation. The initial screening of positive clones was performed by Northern blot. The clone showing higher levels of antiGas1 mRNA was then tested for its resistance to neuronal death induced by staurosporine. In previous it was observed that staurosporine-induced neuronal death involves the induction of endogenous Gas1 and could be prevented by administration to the culture media of the antisense oligonucleotides described herein specific for the amino-terminal of Gas1. As shown in FIG. 8, the DNA laddering associated with the apoptosis induced by staurosporine was not observed in cells stably transfected with antisense Gas1.

Stable expression of human mGluR1 in NB69 cells: protection by antisense Gas1. The suitability of our NB69-ASGas1 cells to support the stable overexpression of potentially lethal proteins was then tested. In pilot experiments, transient transfections in NB69 cells with an expression vector for the human metabotropic GluR1 was performed and the effects on viability after cotransfection with antisenseGas1 expression vector were compared. The results were again evaluated using the two independent tests of cell death; release of lactate dehydrogenase to the culture media at different times and the survival of transfected cells 48 hours after transfection using the expression of LacZ as an interval reporter. The results obtained from a typical experiment performed in triplicate are shown in FIG. 9.

1. A method of inhibiting the lethal effect of expressing an otherwise lethal protein in a cell, said method comprising the steps of:
(a) providing a cell, tissue or organism having
(i) a nucleotide sequence encoding a Gas1 protein, or a functional equivalent, derivative or bioprecursor thereof, which is capable of inducing apoptosis in said cell and
(ii) a further nucleotide sequence encoding a protein which is otherwise lethal to said cell in itself or in response to a lethal stimulus in the presence of Gas1;
(b) inhibiting function and/or expression of said Gas1 protein or functional equivalent, derivative or bioprecursor thereof; and
(c) expressing said sequence encoding said otherwise lethal protein, wherein said protein normally induces the expression or activates either Gas1 protein or a protein in the signal transduction pathway of which Gas1 is a component.

2. A method of identifying compounds which inhibit or enhance expression or activity of proteins which are lethal to a cell, tissue or organism, said method comprising the steps of:
(a) providing a cell, tissue or organism comprising:
i) a nucleotide sequence encoding a Gas1 protein or a functional equivalent, derivative or bioprecursor thereof, which is capable of inducing apoptosis in said cell, and
ii) a further sequence encoding a protein which is otherwise lethal to said cell in itself or in response to a lethal stimulus in the presence of Gas1;
(b) inhibiting function and/or expression of said Gas1 protein or functional equivalent, derivative or bioprecursor thereof or a protein in the apoptotic pathway of which Gas1 is a component;
(c) expressing said sequence encoding said otherwise lethal protein;
(d) contacting said cell with a compound to be tested; and
(e) monitoring the effect of said compound on said otherwise lethal protein compared to an identical cell which has not been contacted with said compound, wherein said proteins normally induce the expression or activate either the Gas1 protein or a protein in the signal transduction pathway of which Gas1 is a component.

3. A method according to claim 2, wherein said expression or activity of Gas1 protein is inhibited by providing a nucleic acid molecule in said cell which is capable of hybridising to mRNA corresponding to Gas1 DNA to prevent expression thereof.

4. A method according to claim 2, wherein said expression or activity of said Gas1 protein is inhibited by inhibiting the expression or activity of a protein in the pathway of which Gas1 is a component.

5. A method according to claim 2, wherein said cell is induced to express said Gas1 protein by contacting said cell with a stimulus that increases intracellular calcium levels in said cell.

6. A method according to claim 5 wherein said cell is induced to express said Gas1 protein by contacting said cell with a suitable compound, such as muristerone.

7. A method according to claim 2, wherein said further sequence encoding said otherwise lethal protein is expressed by providing it on a suitable expression vector.

8. A method according to claim 2, wherein said lethal protein is a highly expressed recombinant protein.

9. A method according to claim 2, wherein said otherwise lethal protein comprises any of a glutamate, NMDA, AMPA or kainate receptor.

10. A method according to claim 9 wherein said glutamate receptors comprises any of a type 1 to 8 metabotropic receptor.

11. A method according to claim 3, wherein said nucleic acid molecule is provided as an oligonucleotide or as a vector including a nucleotide sequence of said nucleic acid molecule.

12. A method according to claim 11 wherein said nucleic acid molecule comprises an oligonucleotide consisting of the nucleotide sequence depicted in SEQ ID NO: 5.

13. A method according to claim 11 wherein said nucleic acid molecule further comprises ribozyme or DNAzyme activity.

14. A method according to claim 2, wherein said Gas1 protein is of mammalian origin.

15. A method according to claim 14 wherein said Gas1 protein is from any of a human, mouse or rat.

16. A method according to claim 14, wherein said Gas1 protein comprises the amino acid sequence depicted in either of SEQ ID NO: 2 or 4 or a functional equivalent, derivative or bioprecursor thereof.

17. A compound identifiable as an inhibitor or an enhancer of expression or activity of an otherwise lethal protein according to the method of claim 2.

18. A pharmaceutical composition comprising a compound according to claim 17 together with a pharmaceutically acceptable carrier, diluent or excipient therefor.

19. A compound according to claim 17 for use as a medicament.

20. Use of a compound identifiable as an enhancer of expression or activity of a lethal protein according to claim 17 in the manufacture of a medicament for treating a disease condition mediated at least in part by underexpression or reduced activity of said otherwise lethal protein or a protein in the pathway of which said otherwise lethal protein is a component.

21. Use of a compound identifiable as an inhibitor of expression or activity of an otherwise lethal protein according to claim 17 in the manufacture of a medicament for treating a disease condition mediated at least in part by overexpression or reduced activity of said otherwise lethal protein or a protein in the pathway of which said otherwise lethal protein is a component.

22. Use method of claim 54 wherein said cell is associated with a disease condition selected from the group consisting of a neurological disorder, a cardiovascular disorder, an autoimmune disorder, a neuroendocrine disorder or cancer.

23. A method of monitoring the severity of a disease condition mediated by cellular apoptosis in a cell, tissue or organism comprising measuring the level of expression or activity of a Gas1 protein or a functional equivalent, derivative or bioprecursor thereof in said cell or tissue or organism

24. A nucleic acid molecule encoding a rat Gas1 protein or a functional equivalent, derivative or bioprecursor thereof, comprising an amino acid sequence according to SEQ ID NO: 2.

25. A nucleic acid molecule encoding a protein capable of inducing apoptosis in a cell comprising an amino acid sequence according to SEQ ID NO: 4 or a nucleic acid molecule complementary thereto.

26. A nucleic acid molecule according to claim 25, which is a DNA sequence.

27. A nucleic acid molecule according to claim 26 which is a cDNA molecule.

28. A nucleic acid molecule according to claim 26, comprising the sequence of nucleotides according to SEQ ID NO: 1.

29. An antisense molecule capable of hybridising to the nucleic acid molecule of claim 25 under conditions of high stringency.

30. An antisense molecule according to claim 29 comprising a sequence of nucleotides according to SEQ ID NO: 3 or 5.

31. A protein encoded by a nucleic acid molecule of SEQ ID NO: 1 or a nucleic acid molecule capable of hybridizing to SEQ ID NO: 1 or its complement under conditions of high stringency.

32. A protein comprising an amino acid sequence illustrated in SEQ ID NO: 2.

33. A protein capable of inducing apoptosis in a cell comprising an amino acid sequence according to SEQ ID NO: 4 or a functional equivalent, derivative or bioprecursor thereof.

34. An expression vector comprising a nucleic acid molecule according to claim 25 or a nucleic acid molecule capable of hybridizing to SEQ ID NO: 1 under conditions of high stringency.

35. An expression vector according to claim 34 wherein said vector is any of a plasmid, virus or phage derived vector.

36. An expression vector according to claim 34 comprising a tissue or cell specific promoter.

37. An expression vector according to claim 34, further comprising a sequence encoding a proapoptotic protein.

38. An expression vector according to claim 34, which is inducible for expression of a protein comprising the amino acid sequence of SEQ ID NO: 2 or a polypeptide capable of inducing apoptosis in a cell.

39. An expression vector according to claim 38 comprising the inducible vector pIND.

40. A host cell, tissue or organism, transformed, transfected or infected with a vector according to claim 34.

41. A method of identifying compounds capable of preventing or accelerating Gas1 mediated cell death comprising the steps of: (a) contacting a cell, tissue or organism expressing Gas1 or a functional equivalent, derivative or bioprecursor thereof capable of inducing apoptosis in a cell with said compound to be tested; and (b) monitoring the effect of said compound on the state of said cell compared to a cell which has not been contacted with said compound.

42. A method according to claim 41, wherein said cell in step (a) comprises a host cell, tissue or organism transformed, transfected or infected with a vector comprising a nucleic acid molecule encoding a protein capable of inducing apoptosiss in a cell comprising an amino acid sequence according to SEQ ID NO: 4 or a nucleic acid molecule capable of hybridizing to SEQ ID NO: 1 under conditions of high stringency.

43. A compound identifiable as an inhibitor or an accelerator of cell death according to the method of claim 42.

44. A pharmaceutical composition comprising a compound according to claim 43, together with a pharmaceutically acceptable carrier, diluent or excipient therefor.

45. A pharmaceutical composition comprising any of a nucleic acid molecule encoding a protein according to SEQ ID NO: 4 or a nucleic acid molecule capable of stringent hybridization thereto; an antisense molecule capable of hybridizing to a nucleic acid molecule encoding a protein according to SEQ ID NO: 4 under conditions of high stringency; or a protein encoded by a nucleic acid molecule capable of hybridizing to SEQ ID NO: 1 under stringent hybridization conditions together with a pharmaceutically acceptable carrier, diluent or excipient therefor.

46. Use of any of a nucleic acid molecule according to any of claims 24 to 28, an antisense molecule according to claim 29 or 30, a protein according to any of claims 31 to 33, a compound according to claim 43 or a pharmaceutical composition according to claim 44, in the manufacture of a medicament for the prevention or treatment of a disease condition mediated at least in part by expression of a Gas1 protein or a functional equivalent, derivative or bioprecursor thereof capable of inducing apoptosis in a cell or a protein in the pathway of which Gas1 is a component.

47. Use according to claim 46 wherein said disease condition is any of a neurological disorder, a cardiovascular disorder, an autoimmune disorder, a neuroendocrine disorder or an oncological disorder.

48. The method of claim 22, wherein said neurological disorder is any of, Parkinson's disease, Alzheimer's disease, Huntington's disease, amyotrophic lateral sclerosis, a neurological condition caused by thrombosis or cerebral trauma.

49. The method of claim 22, where said cardiovascular disorder is a heart attack.

50. The method of claim 22, wherein said autoimmune disorder is multiple sclerosis.

51. The method of claim 22, wherein said neuroendocrine disorder is necrosis of the pituitary gland.

52. An antibody capable of binding to a protein comprising an amino acid sequence according to SEQ ID NO: 2 or SEQ ID NO: 4.

53. A pharmaceutical composition comprising an antibody according to claim 52 together with a pharmaceutically acceptable carrier, diluent or excipient therefor.

54. A method for decreasing the expression of a protein that is lethal to a cell, wherein the protein normally induces the expression or activation of either the Gas1 protein or a protein in the signal transduction pathway of which Gas1 is a component comprising the step of contacting the cell with a therapeutically effective dose of a compound identified by:
(a) providing a cell, tissue or organism comprising:
i) a nucleotide sequence encoding a Gas1 protein or a functional equivalent, derivative or bioprecursor thereof, which is capable of inducing apoptosis in said cell, and
ii) a further sequence encoding a protein which is otherwise lethal to said cell in itself or in response to a lethal stimulus in the presence of Gas1;
(b) inhibiting function and/or expression of said Gas1 protein or functional equivalent, derivative or bioprecursor thereof or a protein in the apoptotic pathway of which Gas1 is a component;
(c) expressing said sequence encoding said otherwise lethal protein;
(d) contacting said cell with a compound to be tested; and
(e) monitoring the effect of said compound on said otherwise lethal protein compared to an identical cell which has not been contacted with said compound.
